(19) United States
(12) Patent Application Publication
(10) Pub. No.: US 2025/0275681 A1
(43) Pub. Date: Sep. 4, 2025

(54) IMAGE CAPTURING APPARATUS, CONTROL METHOD, AND STORAGE MEDIUM

(71) Applicant: CANON KABUSHIKI KAISHA, Tokyo (JP)

(72) Inventors: KIYOTO UEDA, Tokyo (JP); HIROTO OKA, Kanagawa (JP)

(21) Appl. No.: 19/213,282

(22) Filed: May 20, 2025

Related U.S. Application Data
(63) Continuation of application No. PCT/JP2023/040024, filed on Nov. 7, 2023.

(30) Foreign Application Priority Data
Dec. 7, 2022 (JP) 2022-195862

(51) Int. Cl. A61B 5/00; G16H 30/40; H04N 23/65

(52) U.S. Cl. CPC A61B 5/0077; A61B 5/0013; G16H 30/40; H04N 23/651

(57) ABSTRACT

A setting unit sets specific information. An association unit associates the set specific information with an image that has been shot using an image capturing unit. In a case where a medical mode for shooting a medical image is ON, a control unit performs control to in a case where the specific information has not been set, cause an image capturing apparatus to transition to a power saving state when a state where a user has not operated the image capturing apparatus has continued for a predetermined time period, and in a case where the specific information has been set, not cause the image capturing apparatus to transition to the power saving state even if the state where the user has not operated the image capturing apparatus has continued for the predetermined time period.

This application is a Continuation of International Patent Application No. PCT/JP2023/040024, filed Nov. 7, 2023, which claims the benefit of Japanese Patent Application No. 2022-195862, filed Dec. 7, 2022, both of which are hereby incorporated by reference herein in their entirety.

### BACKGROUND

### Field of the Technology

The present disclosure relates to an image capturing apparatus, a control method, and a storage medium.

### Description of the Related Art

A technique is known that records images shot using an image capturing apparatus, such as a digital camera, in association with specific information. For example, in a medical setting, there are cases where shot images are recorded in association with patient information and the like.

Furthermore, a technique is also known that causes an image capturing apparatus to automatically transition to a power saving state (e.g., a sleep state) in a case where a user has not performed an operation for a certain time period, in order to suppress a power consumption of the image capturing apparatus.

PTL 1 discloses a technique to cause a tablet terminal to return to a normal state from a sleep state. According to PTL 1, in a system that manages drug administration with use of a tablet terminal installed in a hospital room, the tablet terminal in a sleep state returns to a normal state upon obtaining a patient ID, an injection drug ID, and a nurse ID.

It takes some time for an image capturing apparatus to return to a normal state from a power saving state. Therefore, if the image capturing apparatus transitions to the power saving state at a timing when a user wishes to perform shooting, shooting cannot be performed promptly. Turning OFF the setting to cause the image capturing apparatus to automatically transition to the power saving state can prevent the image capturing apparatus from transitioning to the power saving state against the user's intention; however, in this case, there is a possibility that a wasteful power consumption increases, and a battery duration is shortened. Conventional techniques, including PTL 1, cannot address such a problem.

### CITATION LIST

### Patent Literature

PTL 1: Japanese Patent Laid-Open No. 2020-140219

### SUMMARY

The present disclosure, in at least one aspect thereof, provides a technique to reduce the possibility that an image capturing apparatus transitions to a power saving state against the user's intention while suppressing an increase in a wasteful power consumption.

According to an aspect of the present disclosure, there is provided an image capturing apparatus including an image capturing unit, comprising: a switching unit configured to switch between ON and OFF of a medical mode of the image capturing apparatus, wherein the medical mode is an operation mode for shooting a medical image; a setting unit configured to set specific information; an association unit configured to associate the set specific information with an image that has been shot using the image capturing unit; and a control unit configured to perform, in a case where the medical mode is ON, control to in a case where the specific information has not been set, cause the image capturing apparatus to transition to a power saving state when a state where a user has not operated the image capturing apparatus has continued for a predetermined time period, and in a case where the specific information has been set, not cause the image capturing apparatus to transition to the power saving state even if the state where the user has not operated the image capturing apparatus has continued for the predetermined time period.

Features of the present disclosure will become apparent from the following description of embodiments with reference to the attached drawings. The following description of embodiments are described by way of example.

### BRIEF DESCRIPTION OF THE DRAWINGS

The accompanying drawings, which are incorporated in and constitute a part of the specification, illustrate embodiments of the disclosure and, together with the description, serve to explain principles of the disclosure.

FIG. 1A is an external view of a digital camera 100.

FIG. 1B is an external view of the digital camera 100.

FIG. 2 is a block diagram showing an exemplary configuration of the digital camera 100.

FIG. 3A is a diagram showing an example of an LV screen for a case where an LV display setting is set to a medical information display.

FIG. 3B is a diagram showing an example of the LV screen for a case where the LV display setting is set to a shooting parameter display.

FIG. 4A is a flowchart showing processing executed by the digital camera 100 in a case where a medical mode is ON.

FIG. 4B is a flowchart showing processing executed by the digital camera 100 in a case where the medical mode is ON.

FIG. 4C is a flowchart showing processing executed by the digital camera 100 in a case where the medical mode is ON.

FIG. 4D is a flowchart showing processing executed by the digital camera 100 in a case where the medical mode is ON.

FIG. 5A is a diagram showing an example of a barcode obtainment screen displayed in a patient information obtainment mode and a photographer information obtainment mode.

FIG. 5B is a diagram showing an example of a confirmation screen of patient information (in the case of the patient information obtainment mode) or photographer information (in the case of the photographer information obtainment mode) corresponding to a shot barcode.

FIG. 5C is a diagram showing an example of an error screen displayed in a case where the obtainment (reception) of the patient information or the photographer information has failed.

FIG. 6A is a flowchart of processing in the patient information obtainment mode and the photographer information obtainment mode.

FIG. 6B is a flowchart of processing in the patient information obtainment mode and the photographer information obtainment mode.

FIG. 7A is a diagram showing an example of an area information selection screen.

FIG. 7B is a diagram showing an example of an error screen.

FIG. 8 is a flowchart of processing in an area information obtainment mode.

FIG. 9 is a flowchart of processing in a lesion shooting mode.

FIG. 10 is a diagram showing an example of a confirmation screen for a lesion image.

FIG. 11A is a flowchart of processing in a lesion shooting end mode.

FIG. 11B is a flowchart of processing in the lesion shooting end mode.

FIG. 12 is a flowchart of processing for switching between ON and OFF of the medical mode.

FIG. 13A is a diagram showing an example of a list display screen of power saving items.

FIG. 13B is a diagram showing an example of a detailed setting screen of a power saving item corresponding to “communication OFF”.

### DESCRIPTION OF THE EMBODIMENTS

Hereinafter, embodiments will be described in detail with reference to the attached drawings. Note, the following embodiments are not intended to limit the scope of the claims. Multiple features are described in the embodiments, but it is not the case that all such features are required, and multiple such features may be combined as appropriate. Furthermore, in the attached drawings, the same reference numerals are given to the same or similar configurations, and redundant description thereof is omitted.

### First Embodiment

### External View of Digital Camera 100

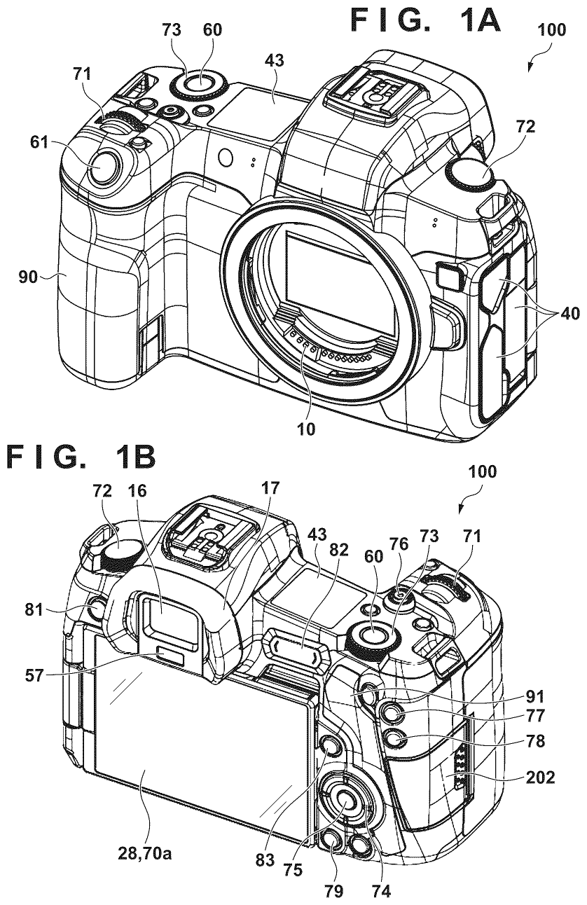

FIG. 1A and FIG. 1B are external views of a digital camera 100, which is one example of an image capturing apparatus. FIG. 1A is a front perspective view of the digital camera 100, and FIG. 1B is a rear perspective view of the digital camera 100.

A display unit 28 is a display unit provided on a rear surface of the digital camera 100, and displays images and various types of information. A touch panel 70a can detect a touch operation on a display surface (a touch operation surface) of the display unit 28. An out-of-viewfinder display unit 43 is a display unit provided on a top surface of the digital camera 100, and displays a variety of setting values of the digital camera 100, including a shutter speed and a diaphragm. A shutter button 61 is an operation member for issuing a shooting instruction. A mode changing switch 60 is an operation member for changing among various types of modes. Terminal covers 40 are covers that protect connectors (not shown) for connecting connection cables intended to connect the digital camera 100 to an external device.

A main electronic dial 71 is a rotary operation member, and rotating the main electronic dial 71 can, for example, change such setting values as the shutter speed and the diaphragm. A power source switch 72 is an operation member that switches between ON and OFF of a power source of the digital camera 100. A sub electronic dial 73 is a rotary operation member, and rotating the sub electronic dial 73 enables, for example, moving of a selection frame (cursor) and an image jump. A four-direction key 74 is configured in such a manner that upper, lower, left, and right portions thereof can each be pushed. The digital camera 100 executes processing corresponding to a pushed portion of the four-direction key 74. A SET button 75 is a push button, and is mainly used in, for example, deciding on a selected item.

A moving image button 76 is used to issue an instruction for starting and stopping the shooting (recording) of moving images. An AE lock button 77 is a push button, and depressing the AE lock button 77 in a shooting standby state can fix an exposure state. A magnification button 78 is an operation button for switching between ON and OFF of a magnification mode during live-view display (LV display) in a shooting mode. A live-view image (LV image) can be magnified or reduced by operating the main electronic dial 71 while the magnification mode is ON. In a reproduction mode, the magnification button 78 functions as an operation button for magnifying a reproduced image and increasing the magnification rate thereof. A reproduction button 79 is an operation button for switching between the shooting mode and the reproduction mode. Depressing the reproduction button 79 during the shooting mode causes the digital camera 100 to transition to the reproduction mode; as a result, among the images recorded in a recording medium 200 (described later), the newest image can be displayed on the display unit 28. A menu button 81 is a push button that is used to perform an instruction operation for displaying a menu screen; when the menu button 81 has been pushed, the display unit 28 displays the menu screen on which various types of settings can be configured. A user can intuitively configure various types of settings with use of the menu screen displayed on the display unit 28, the four- direction key 74, and the SET button 75. A display switching button 83 is used to switch a display setting of the display unit 28.

A touch bar 82 (multi-function bar/M-Fn bar) is a line-like touch operation member (line touch sensor) that can accept a touch operation. The touch bar 82 is located at a position where it can be touch-operated (can be touched) by a thumb of a right hand in a state where a grip unit 90 is grasped by the right hand (in a state where it is grasped by a little finger, a ring finger, and a middle finger of the right hand) in such a manner that the shutter button 61 can be depressed by a pointing finger of the right hand. That is to say, the touch bar 82 is located at a position where it can be operated in a state where an eye is looking through a viewfinder while in proximity to an eye proximity unit 16 and the digital camera is held in such a manner that the shutter button 61 can be depressed at any time (a shooting posture). The touch bar 82 is an acceptance unit that can accept a tap operation (an operation of touching and then releasing within a predetermined time period without moving a touch position), a leftward/rightward slide operation (an operation of touching and then moving a touch position while maintaining the touch), and the like on the touch bar 82. The touch bar 82 is an operation member different from the touch panel 70a, and does not have a display function.

A communication terminal 10 is a communication terminal intended for the digital camera 100 to communicate with an attachable/removable lens unit 150 (described later). The eye proximity unit 16 is an eye proximity unit for an eye proximity viewfinder 17 (look-through viewfinder), and the user can visually recognize a video displayed on an internal EVF 29 (Electronic View Finder) via the eye proximity unit 16. An eye proximity detection unit 57 is an eye proximity detection sensor that detects whether an eye of the user (photographer) is in proximity to the eye proximity unit 16. A cover 202 is a cover for a slot in which the recording medium 200 (described later) is stored. The grip unit 90 is a holding unit with a shape that is easily grasped by the right hand while the user is holding the digital camera 100. The shutter button 61 and the main electronic dial 71 are located at positions where they can be operated by the pointing finger of the right hand in a state where the digital camera 100 is held while the grip unit 90 is grasped by the little finger, the ring finger, and the middle finger of the right hand. Furthermore, the sub electronic dial 73 and the touch bar 82 are located at positions where they can be operated by the thumb of the right hand in the same state. A thumb rest unit 91 (thumb standby position) is a grip member provided at a location which is on the rear surface side of the digital camera 100, and at which the thumb of the right hand grasping the grip unit 90 easily rests in a state where the thumb is not operating any operation member. The thumb rest unit 91 is made up of, for example, a rubber member for increasing a holding force (grip feeling).

### Block Diagram of Digital Camera 100

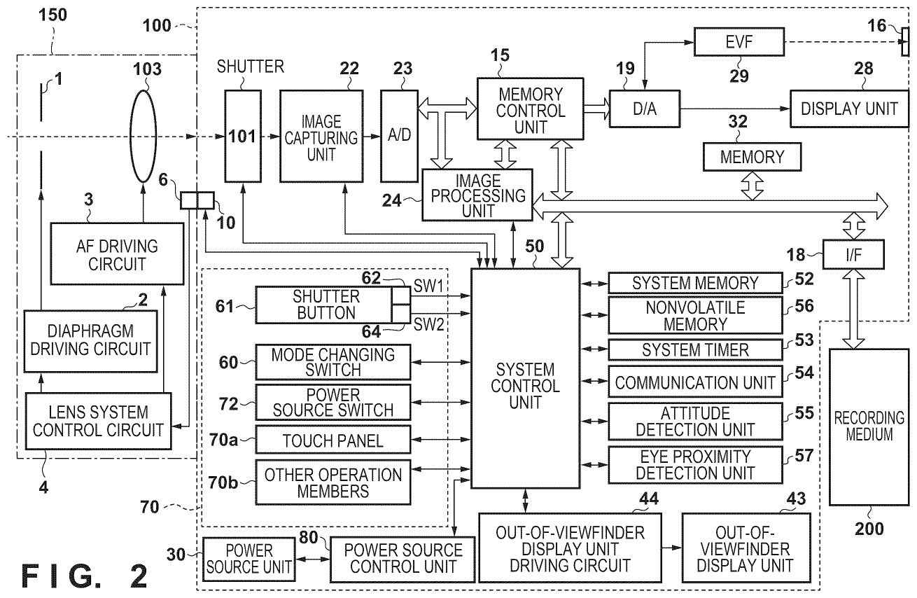

FIG. 2 is a block diagram showing an exemplary configuration of the digital camera 100. The lens unit 150 is an interchangeable lens unit provided with a shooting lens (lens 103). The lens 103 is normally composed of a plurality of lenses, but is represented as only one lens in FIG. 2 for simplicity.

A communication terminal 6 is a communication terminal intended for the lens unit 150 to communicate with the digital camera 100 side, and a communication terminal 10 is a communication terminal intended for the digital camera 100 to communicate with the lens unit 150 side. The lens unit 150 communicates with a system control unit 50 via these communication terminals 6 and 10. Also, the lens unit 150 causes an internal lens system control circuit 4 to control a diaphragm 1 via a diaphragm driving circuit 2. Furthermore, the lens unit 150 achieves focus by causing the lens system control circuit 4 to displace the position of the lens 103 via an AF driving circuit 3.

A shutter 101 is a focal-plane shutter that can freely control an exposure time period of an image capturing unit 22 under control of the system control unit 50.

The image capturing unit 22 is an image capturing element (image sensor) composed of, for example, a CCD or CMOS element that converts an optical image into electrical signals. The image capturing unit 22 may include an image capturing plane phase-difference sensor that outputs defocus amount information to the system control unit 50. An A/D converter 23 converts analog signals output from the image capturing unit 22 into digital signals.

An image processing unit 24 executes predetermined processing (e.g., pixel interpolation processing, resize processing like reduction, and color conversion processing) with respect to data from the A/D converter 23, or data from a memory control unit 15. Also, The image processing unit 24 executes predetermined computation processing with use of captured image data. The system control unit 50 performs exposure control and range-finding control on the basis of the computation result obtained by the image processing unit 24. As a result, for example, autofocus (AF) processing, automatic exposure (AE) processing, and preliminary flash emission (EF) processing of the through-the-lens (TTL) method are executed. The image processing unit 24 further executes predetermined computation processing with use of captured image data, and executes auto white balance (AWB) processing of the TTL method on the basis of the obtained computation result.

Output data from the A/D converter 23 is written into a memory 32 via the image processing unit 24 and the memory control unit 15. Alternatively, output data from the A/D converter 23 is written into the memory 32 via the memory control unit 15 without intervention of the image processing unit 24. The memory 32 stores image data that has been obtained by the image capturing unit 22 and converted into digital data by the A/D converter 23, and image data to be displayed on the display unit 28 and the EVF 29. The memory 32 has a storage capacity sufficient to store a predetermined number of still images, and moving images and audio of a predetermined duration.

Furthermore, the memory 32 also functions as a memory for image display (video memory). A D/A converter 19 converts image data for display stored in the memory 32 into analog signals, and supplies the analog signals to the display unit 28 and the EVF 29. In this way, image data for display that has been written into the memory 32 is displayed by the display unit 28 and the EVF 29 via the D/A converter 19. Each of the display unit 28 and the EVF 29 is a display, such as an LCD and organic EL, and performs display in accordance with analog signals from the D/A converter 19. Digital signals that have undergone A/D conversion in the A/D converter 23 and have been accumulated in the memory 32 are converted into analog signals by the D/A converter 19, and sequentially transferred to and displayed by the display unit 28 or the EVF 29; as a result, live-view display (LV display) can be performed. Hereinafter, images that are displayed in the form of live-view display will be referred to as live-view images (LV images).

The system control unit 50 is a control unit composed of at least one processor and/or at least one circuit, and controls an entirety of the digital camera 100. The system control unit 50 realizes each process of the present embodiment, which will be described later, by executing a program recorded in a nonvolatile memory 56. Furthermore, the system control unit 50 also performs display control by controlling the memory 32, the D/A converter 19, the display unit 28, the EVF 29, and so forth.

A system memory 52 is, for example, a RAM. The system control unit 50 deploys constants and variables for the operations of the system control unit 50, the program that has been read out from the nonvolatile memory 56, and the like to the system memory 52.

The nonvolatile memory 56 is an electrically erasable and recordable memory, and is, for example, an EEPROM and the like. The constants for the operations of the system control unit 50, the program, and the like are recorded in the nonvolatile memory 56. The program mentioned here denotes a program for executing processing of various types of flowcharts, which will be described later in the present embodiment.

A system timer 53 is a time measurement unit that measures the time periods used in various types of control, and the time of a built-in clock.

A communication unit 54 transmits/receives video signals and audio signals to/from an external device that is connected wirelessly or via a wired cable. The communication unit 54 is also connectable to a wireless Local Area Network (wireless LAN) and the Internet. Furthermore, the communication unit 54 can communicate with an external device also via Bluetooth® and Bluetooth Low Energy. The communication unit 54 can transmit images captured by the image capturing unit 22 (including LV images) and images recorded in the recording medium 200, and can receive image data and other various types of information from an external device.

An attitude detection unit 55 detects an attitude of the digital camera 100 relative to the gravity direction. Based on the attitude detected by the attitude detection unit 55, it is possible to distinguish whether an image shot by the image capturing unit 22 is an image that was shot while the digital camera 100 was held in a landscape orientation or an image that was shot while the digital camera 100 was held in a portrait orientation. The system control unit 50 can add direction information corresponding to the attitude detected by the attitude detection unit 55 to an image file of an image captured by the image capturing unit 22. Furthermore, the system control unit 50 can rotate an image and record the resultant image on the basis of direction information corresponding to the attitude detected by the attitude detection unit 55. For example, an acceleration sensor or a gyro sensor can be used as the attitude detection unit 55. A motion of the digital camera 100 (e.g., whether the digital camera 100 is panned, tilted, lifted, or stationary) can also be detected using the acceleration sensor or the gyro sensor acting as the attitude detection unit 55.

The eye proximity detection unit 57 is an eye proximity detection sensor that detects approaching (eye proximity) and separation (eye separation) of an eye (object) to and from and the eye proximity unit 16 of the eye proximity viewfinder 17 (approach detection). The system control unit 50 switches between display (a displaying state) and non-display (a non-displaying state) of the display unit 28 and the EVF 29 in accordance with the state detected by the eye proximity detection unit 57. More specifically, for example, in a case where the setting of switching of a display destination is automatic switching during the shooting standby state, the system control unit 50 sets the display unit 28 as the display destination and turns ON its display, and causes the EVF 29 to display nothing, while the eye is not in proximity. Also, the system control unit 50 sets the EVF 29 as the display destination and turns ON its display, and causes the display unit 28 to display nothing, while the eye is in proximity.

For example, an infrared proximity sensor can be used as the eye proximity detection unit 57, and approaching of some sort of object to the eye proximity unit 16 of the eye proximity viewfinder 17, in which the EVF 29 is built, can be detected. In a case where an object has approached, infrared light projected from a light projection unit (not shown) of the eye proximity detection unit 57 is reflected by the object and received by a light receiving unit (not shown) of the infrared proximity sensor. It is also possible to distinguish at what distance from the eye proximity unit 16 the approaching object is situated (an eye proximity distance) on the basis of the amount of the received infrared light. In this way, the eye proximity detection unit 57 performs eye proximity detection for detecting a distance at which the object is approaching the eye proximity unit 16. It is assumed that eye proximity is detected in a case where an approaching object at a predetermined distance or less from the eye proximity unit 16 has been detected in a non-eye proximity state (non-approaching state). It is assumed that eye separation is detected in a case where an object whose approach was detected has separated by a predetermined distance or more in an eye proximity state (approaching state). A threshold for detection of eye proximity and a threshold for detection of eye separation may be different from each other due to, for example, provision of hysteresis and the like. Furthermore, it is assumed that a detection of eye proximity is followed by the eye proximity state, until eye separation is detected. It is assumed that a detection of eye separation is followed by the non-eye proximity state, until eye proximity is detected. Note that the infrared proximity sensor is one example, and another sensor may be used as the eye proximity detection unit 57 as long as it can detect a state that can be regarded as eye proximity.

The out-of-viewfinder display unit 43 displays a variety of setting values of the camera, including the shutter speed and the diaphragm, via an out-of-viewfinder display unit driving circuit 44.

A power source control unit 80 is composed of a battery detection circuit, a DC-DC converter, a switch circuit that changes a block to which current is supplied, and the like, and detects whether a battery is loaded, a battery type, and a remaining battery level, for example. Also, the power source control unit 80 controls the DC-DC converter on the basis of the results of the foregoing detection and an instruction from the system control unit 50, and supplies a necessary voltage for a necessary time period to each unit, including the recording medium 200. A power source unit 30 is composed of, for example, a primary battery like an alkaline battery or a lithium battery, a secondary battery like a NiCd battery, a NiMH battery, or a Li battery, or an AC adapter.

A recording medium I/F 18 is an interface with the recording medium 200, which is a memory card, a hard disk, and the like. The recording medium 200 is a recording medium, such as a memory card, for recording shot images, and is composed of a semiconductor memory, a magnetic disk, and the like.

An operation unit 70 is an input unit that accepts an operation from the user (a user operation), and is used to input various types of operational instructions to the system control unit 50. As shown in FIG. 2, the operation unit 70 includes the shutter button 61, the mode changing switch 60, the power source switch 72, the touch panel 70a, and other operation members 70b. Other operation members 70b include, for example, the main electronic dial 71, the sub electronic dial 73, the four-direction key 74, the SET button 75, the moving image button 76, the AE lock button 77, the magnification button 78, the reproduction button 79, the menu button 81, and the touch bar 82.

The shutter button 61 includes a first shutter switch 62 and a second shutter switch 64. The first shutter switch 62 is turned ON and generates a first shutter switch signal SW1 halfway through an operation performed on the shutter button 61, that is to say, when the shutter button 61 is half-pressed (a shooting preparation instruction). In response to the first shutter switch signal SW1, the system control unit 50 starts shooting preparation operations, such as autofocus (AF) processing, automatic exposure (AE) processing, auto white balance (AWB) processing, and preliminary flash emission (EF) processing.

The second shutter switch 64 is turned ON and generates a second shutter switch signal SW2 upon completion of the operation performed on the shutter button 61, that is to say, when the shutter button 61 is full-pressed (a shooting instruction). In response to the second shutter switch signal SW2, the system control unit 50 starts a sequence of operations of shooting processing, from readout of signals from the image capturing unit 22 to writing of a captured image as an image file into the recording medium 200.

The mode changing switch 60 changes an operation mode of the system control unit 50 to one of a still image shooting mode, a moving image shooting mode, a reproduction mode, and so forth. Modes included in the still image shooting mode include an auto shooting mode, an auto scene distinction mode, a manual mode, a diaphragm priority mode (Av mode), a shutter speed priority mode (Tv mode), and a program AE mode (P mode). They also include various types of scene modes in which shooting settings vary for each shooting scene, a custom mode, and so forth. The mode changing switch 60 enables the user to change directly to one of these modes. Alternatively, it is permissible to adopt a configuration in which, after the user has changed to a screen of a list of shooting modes with use of the mode changing switch 60, the user can selectively change to one of the displayed plurality of modes with use of another operation member. Similarly, the moving image shooting mode may also include a plurality of modes.

The touch panel 70a is a touch sensor that detects various types of touch operations on the display surface of the display unit 28 (an operation surface of the touch panel 70a). The touch panel 70a and the display unit 28 can be configured integrally. For example, the touch panel 70a is configured so that a light transmittance thereof does not interfere with display of the display unit 28, and is attached to a top layer of the display surface of the display unit 28. Also, input coordinates on the touch panel 70a are associated with display coordinates on the display surface of the display unit 28. This makes it possible to provide a graphical user interface (GUI) via which the user can operate a screen displayed on the display unit 28, as if directly.

The system control unit 50 can detect the following operations or states with respect to the touch panel 70a.
Newly touching the touch panel 70a with a finger or a stylus that was not touching the touch panel 70a, that is to say, the start of a touch (hereinafter referred to as “touch-down”)
A state where a finger or a stylus is touching the touch panel 70a (hereinafter referred to as “touch-on”)
Movement of a finger or a stylus while it is touching the touch panel 70a (hereinafter referred to as “touch-move”)
Separation (releasing) of a finger or a stylus that was touching the touch panel 70a from the touch panel 70a, that is to say, the end of a touch (hereinafter referred to as “touch-up”)
A state where nothing is touching the touch panel 70a (hereinafter referred to as “touch-off”)

When a touch-down is detected, a touch-on is detected at the same time. After a touch-down, a touch-on normally continues to be detected as long as no touch-up is detected. Also in a case where a touch-move is detected, a touch-on is detected at the same time. Even when a touch-on is detected, a touch-move is not detected as long as a touch position does not move. A touch-off follows the detection of a touch-up of every finger or stylus that was touching.

The system control unit 50 is notified of these operations and states, as well as positional coordinates at which a finger or a stylus is touching the touch panel 70a, via an internal bus. Then, the system control unit 50 judges what kind of operation (touch operation) has been performed on the touch panel 70a on the basis of information it has been notified of. Regarding a touch-move, a moving direction of a finger or a stylus moving on the touch panel 70a can also be judged for each of a vertical component and a horizontal component on the touch panel 70a on the basis of a change in positional coordinates. It is assumed that, in a case where a touch-move of a predetermined distance or longer has been detected, it is judged that a slide operation has been performed. An operation of quickly moving a finger by some distance while the finger is touching the touch panel 70a and then releasing the finger is called a flick. In other words, a flick is a quick tracing operation involving a swipe of a finger on the touch panel 70a. It can be judged that a flick has been performed when a touch-up is detected immediately after the detection of a touch-move of a predetermined distance or longer at a predetermined speed or faster (it can be judged that a flick has been performed following a slide operation). Furthermore, a touch operation of bringing the touch positions close to each other and a touch operation of moving the touch positions away from each other, while touching a plurality of locations (e.g., two points) at the same time (performing a multi-touch), are referred to as a pinch-in and a pinch-out, respectively. A pinch-out and a pinch-in are collectively referred to as a pinch operation (or simply a pinch). The touch panel 70a may be of any type included among a variety of types of touch panels, such as a resistive film type, a capacitance type, a surface acoustic wave type, an infrared type, an electromagnetic induction type, an image recognition type, and an optical sensor type. There are a type in which a touch is detected on the occurrence of contact with the touch panel, and a type in which a touch is detected when a finger or a stylus has approached the touch panel; either type may be used.

### Basic Operations of Digital Camera 100

Basic operations of the digital camera 100 according to the present embodiment will be described. In the following description, it is assumed that the digital camera 100 is used to shoot a lesion image, which is one example of a medical image. Also, it is assumed that the digital camera 100 is configured to associate medical information with a lesion image. Medical information includes, for example, at least one of patient information (information related to a patient), photographer information (information related to a photographer), and area information (information related to a shooting target area of a patient). However, images shot by the digital camera 100 are not limited to lesion images, and information that the digital camera 100 associates with lesion images is not limited to medical information. The technique ideas of the present embodiment are applicable to an image capturing apparatus that associates specific information with shot images, regardless of the types of images and information.

The digital camera 100 is configured to communicate with an external system including an external device via the communication unit 54. In the following description, it is assumed that the external system is a medical system. The digital camera 100 is configured to be capable of switching between ON (enabled) and OFF (disabled) of a medical mode (a predetermined operation mode). The digital camera 100 includes a patient information obtainment mode, a photographer information obtainment mode, an area information obtainment mode, a lesion shooting mode, and a lesion shooting end mode, as operation modes in a state where the medical mode is ON.

In the patient information obtainment mode, the digital camera 100 transmits image data including a code (e.g., a one-dimensional code or a two-dimensional code), which has been obtained through shooting or an LV operation, to the external device via the communication unit 54. The digital camera 100 receives patient information (e.g., an ID, a name, a sex, an age, and the like) corresponding to the code included in the transmitted image data from the external device via the communication unit 54, and stores the patient information into the system memory 52. Note that the digital camera 100 may obtain code information by analyzing the code included in the image data, and transmit the code information, in place of the image data, to the external device. In the following description, it is assumed that image data including a barcode (a barcode image) is obtained as the image data including the code in the patient information obtainment mode.

In the photographer information obtainment mode, the digital camera 100 transmits image data including a code (e.g., a one-dimensional code or a two-dimensional code), which has been obtained through shooting or an LV operation, to the external device via the communication unit 54. The digital camera 100 receives photographer information (e.g., an ID, a name, a clinical department, and the like) corresponding to the code included in the transmitted image data from the external device via the communication unit 54, and stores the photographer information into the system memory 52. Note that the digital camera 100 may obtain code information by analyzing the code included in the image data, and transmit the code information, in place of the image data, to the external device. In the following description, it is assumed that image data including a barcode (a barcode image) is obtained as the image data including the code in the photographer information obtainment mode.

In the area information obtainment mode, the digital camera 100 obtains area list information (an area list) from the external device via the communication unit 54, and stores the area list information into the system memory 52. Once the user has selected and decided on an area corresponding to a lesion from the area list, the digital camera 100 stores information indicating the selected area (area information) into the system memory 52.

In the lesion shooting mode, the digital camera 100 obtains image data of a subject (a lesion of a patient) in accordance with a shooting operation of the user. The digital camera 100 links the obtained image data with the medical information including patient information, photographer information, and area information stored in the system memory 52, and saves the same in the recording medium 200. Also, the digital camera 100 transmits the image data and the medical information linked therewith from the communication unit 54 to the external device. A method of linking the image data with the medical information is not limited in particular. For example, the digital camera 100 can embed the medical information in a part of the image data. Alternatively, the digital camera 100 may save the medical information in the recording medium 200 as a file different from a file of the image data, and link the file of the medical information with the file of the image data.

In the lesion shooting end mode, the digital camera 100 displays thumbnail images of the image data shot in the lesion shooting mode, and the medical information linked with the image data. The user confirms the displayed thumbnail images and medical information, and selects whether to transfer the image data and the medical information to the medical system. Also, the digital camera 100 clears the medical information including the patient information, the photographer information, and the area information held in the system memory 52, thereby cancelling the setting of the medical information.

### Examples of Live-View Screen (LV Screen)

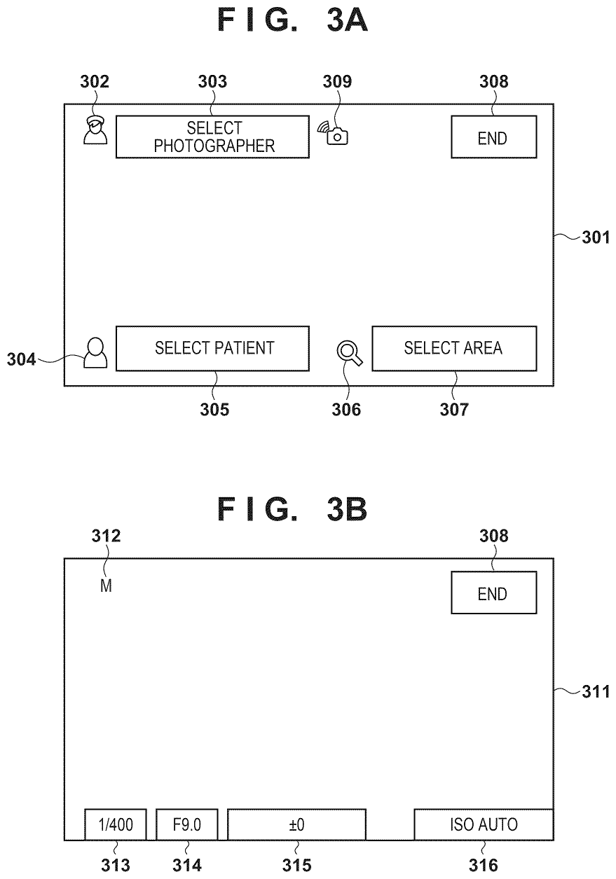

FIG. 3A and FIG. 3B are diagrams showing examples of an LV screen (a shooting standby screen) displayed during a shooting standby in a case where the medical mode is ON. The digital camera 100 displays the LV screen in accordance with an LV display setting. The LV display setting includes a medical information display and a shooting parameter display. The user can switch between the medical information display and the shooting parameter display by depressing the display switching button 83. Also, the digital camera 100 may switch the LV display setting to the medical information display when the user has half-pressed the shutter button 61.

FIG. 3A is a diagram showing an example of the LV screen for a case where the LV display setting is set to the medical information display. 301 is a display region of the LV image. Icons, text boxes, and buttons indicated by 302 to 309 are displayed while being superimposed on the LV image.

302 is an icon indicating a photographer, and indicates that a text box (a photographer information frame 303) next to it displays information of the photographer. After photographer information has been obtained in the photographer information obtainment mode, the digital camera 100 may switch the icon 302 to an icon with a design tailored to the photographer information.

303 is the photographer information frame. When the user has touched the photographer information frame 303 (or when the user has moved a selection frame to the photographer information frame 303 through a dial operation and depressed the SET button 75), the digital camera 100 transitions to the photographer information obtainment mode. The digital camera 100 displays photographer information (including a photographer name) obtained in the photographer information obtainment mode in the photographer information frame 303.

Note that in the present specification, in a case where some sort of processing is executed through the “dial operation”, the “dial operation” refers to an operation on a dial to which this processing has been assigned among the main electronic dial 71 and the sub electronic dial 73 included in the digital camera 100. For example, in a case where processing for moving the selection frame on the LV screen corresponding to the medical information display is assigned to the sub electronic dial 73, the “dial operation” in “the user has moved the selection frame to the photographer information frame 303 through the dial operation” corresponds to an operation on the sub electronic dial 73.

304 is an icon indicating a patient, and indicates that a text box (a patient information frame 305) next to it displays information of the patient. After patient information has been obtained in the patient information obtainment mode, the digital camera 100 may change the icon 304 to an icon with a design tailored to the patient information. As one example, the icon may be switched in accordance with the patient's sex and age.

305 is the patient information frame. When the user has touched the patient information frame 305 (or when the user has moved the selection frame to the patient information frame 305 through a dial operation and depressed the SET button 75), the digital camera 100 transitions to the patient information obtainment mode. The digital camera 100 displays the patient information (including a patient name) obtained in the patient information obtainment mode in the patient information frame 305. As one example, the digital camera 100 may display a patient name, a sex, and an age.

306 is an icon indicating an area, and indicates that a text box (an area information frame 307) next to it displays information on area selection. After area information has been obtained in the area information obtainment mode, the digital camera 100 may switch the icon 306 to an icon with a design tailored to the area information.

307 is the area information frame. When the user has touched the area information frame 307 (or when the user has moved the selection frame to the area information frame 307 through a dial operation and depressed the SET button 75), the digital camera 100 transitions to the area information obtainment mode. The digital camera 100 displays area information (including an area name) obtained in the area information obtainment mode in the area information frame 307.

When the user has full-pressed the shutter button 61, the digital camera 100 transitions to the lesion shooting mode. In the lesion shooting mode, the digital camera 100 performs shooting, associates shot image data with medical information, and transmits the same to the external device.

308 is an end button. The user depresses the end button 308 when shooting of a necessary number of images has ended. When the end button 308 has been depressed, the digital camera 100 transitions to the lesion shooting end mode. The user selects whether to transfer lesion images to the medical system. In a case where the transfer of the lesion images to the medical system has been selected, the digital camera 100 transfers the lesion images to the medical system. Also, the digital camera 100 clears the photographer information, the patient information, and the area information.

309 is an icon indicating a communication state. The icon 309 indicates whether the digital camera 100 is unconnected to the external device or is currently connected to the external device.

FIG. 3B is a diagram showing an example of the LV screen for a case where the LV display setting is set to the shooting parameter display.

311 is a display region of the LV image. 312 indicates a shooting mode. 313 indicates a setting value of the shutter speed. 314 indicates an f-number. 315 indicates an exposure correction value. 316 indicates an ISO value.

### Processing in Medical Mode

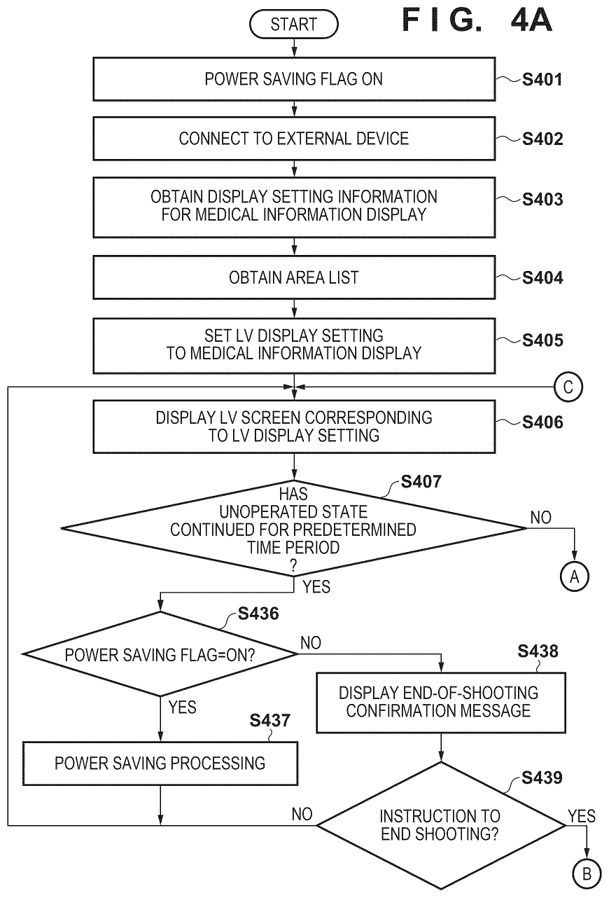

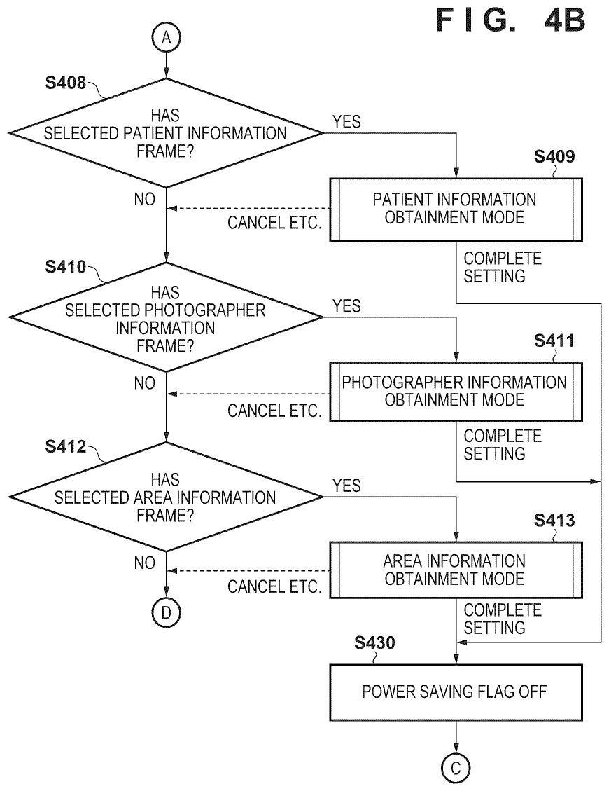

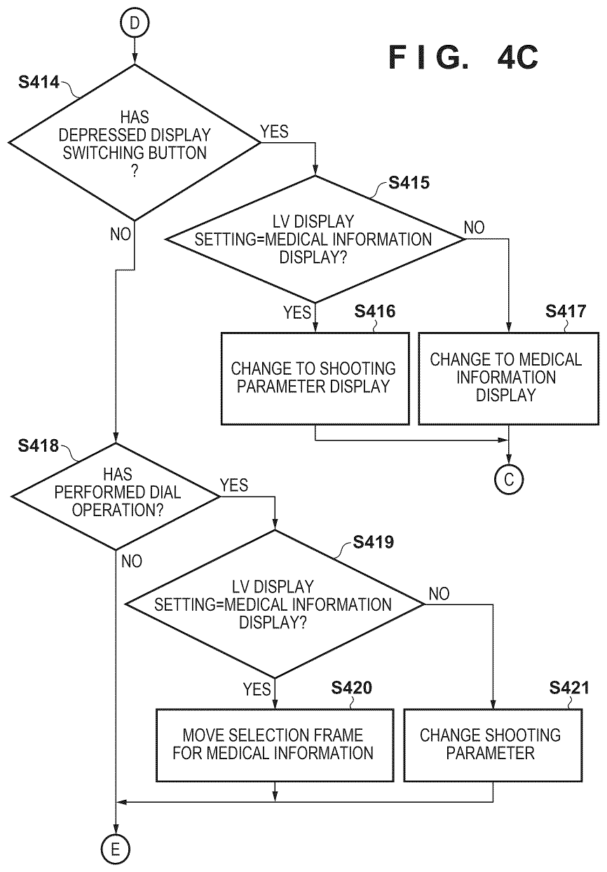

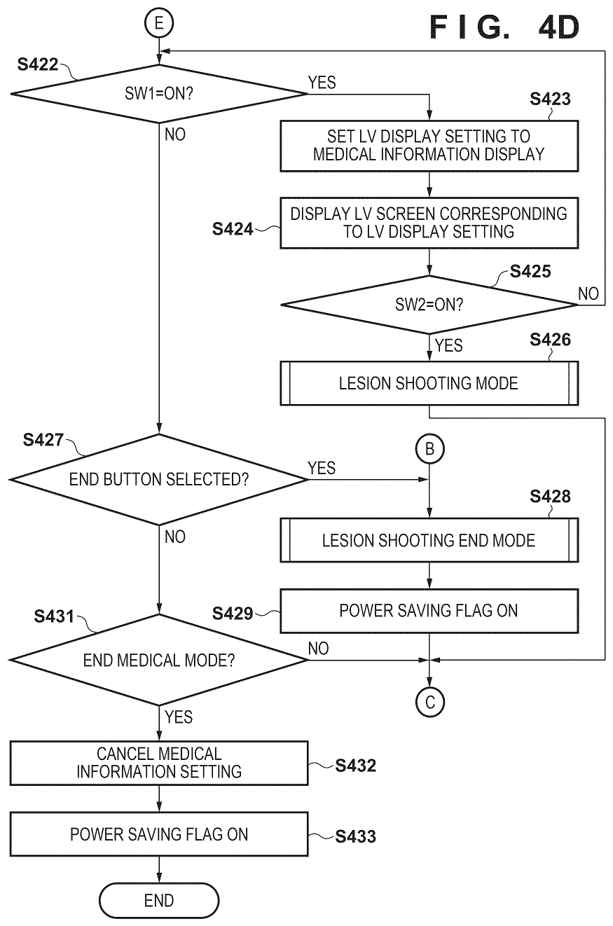

FIGS. 4A to 4D are a flowchart showing processing executed by the digital camera 100 in a case where the medical mode is ON. Processing of the present flowchart is executed in a case where the power source of the digital camera 100 has been turned ON in a state where the medical mode is set to be ON, or in a case where the user has switched the setting of the medical mode from OFF to ON in a state where the power source of the digital camera 100 is ON. Processing of each step in the present flowchart is realized by the system control unit 50 deploying the program stored in the nonvolatile memory 56 to the system memory 52 and executing the program, unless specifically stated otherwise. In the following description, the user (photographer) is, for example, a doctor, a nurse, or the like.

In S401, the system control unit 50 turns ON a power saving flag.

In S402, the system control unit 50 connects to an external device via the communication unit 54. The mode of connection is assumed to be wireless connection, but may be wired connection.

In S403, the system control unit 50 obtains display setting information for the medical information display (FIG. 3A) from the external device. The display setting information designates display or non-display with respect to each of photographer information (reference signs 302 and 303), patient information (reference signs 304 and 305), and area information (reference signs 306 and 307) on the medical information display.

In S404, the system control unit 50 obtains an area list from the external device. The area list includes candidates for areas that can be selected by the user in the area information obtainment mode.

In S405, the system control unit 50 sets the LV display setting to the medical information display. Therefore, the display format shown in FIG. 3A is used as an initial display format of the LV screen.

In S406, the system control unit 50 displays the LV screen corresponding to the LV display setting on the display unit 28. In a case where the LV display setting is the medical information display, the LV screen shown in FIG. 3A is displayed; in a case where the LV display setting is the shooting parameter display, the LV screen shown in FIG. 3B is displayed.

In S407, the system control unit 50 judges whether a state where the user has not operated the digital camera 100 (an unoperated state) has continued for a predetermined time period. In a case where the unoperated state has continued for the predetermined time period, processing proceeds to S436; otherwise, processing proceeds to S408.

In S408, the system control unit 50 judges whether the user has selected the patient information frame 305 on the LV screen. The selection of the patient information frame 305 is made by touching the patient information frame 305, or by moving the selection frame to the patient information frame 305 through a dial operation or a direction key operation and depressing the SET button 75. In a case where the patient information frame 305 has been selected, processing proceeds to S409; otherwise, processing proceeds to S410.

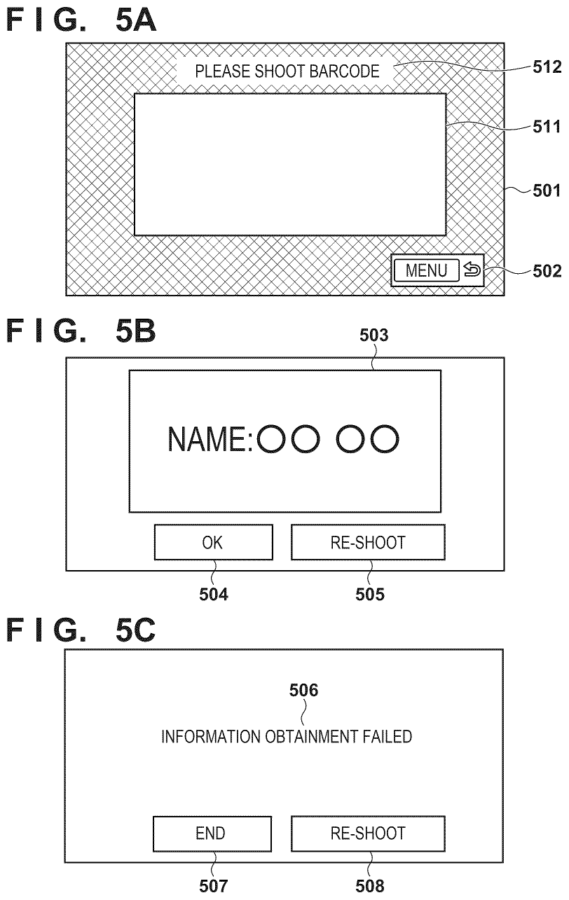

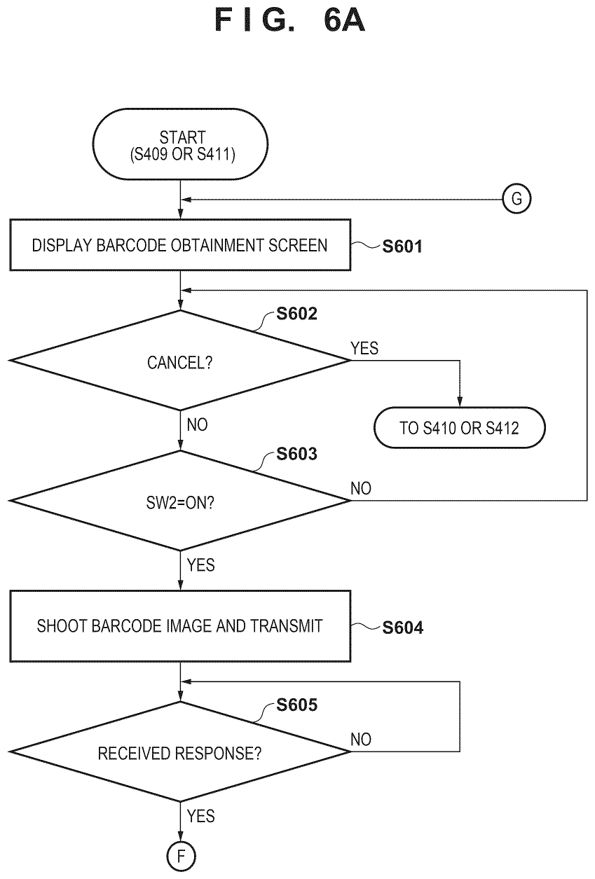

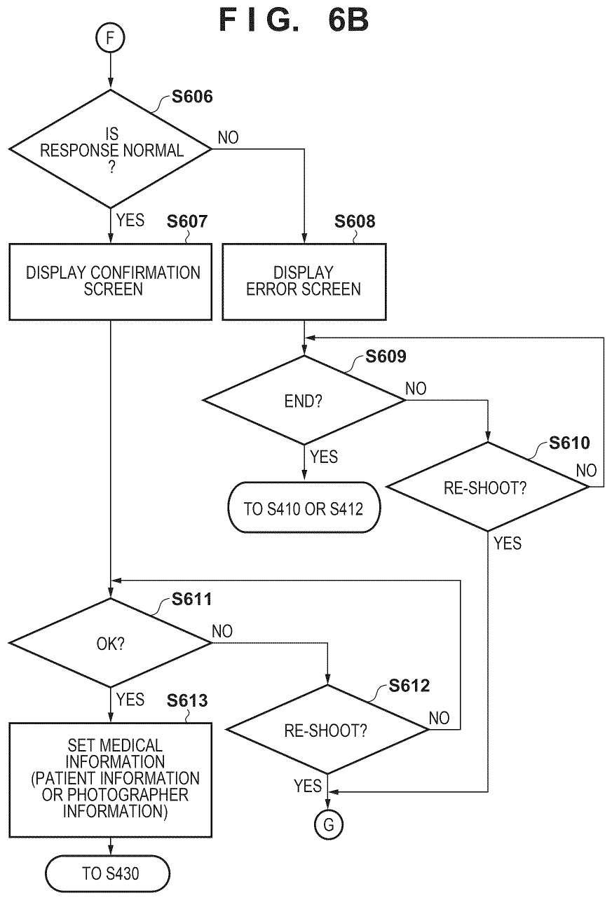

In S409, the system control unit 50 executes processing in the patient information obtainment mode. Through this processing, medical information (here, patient information) to be associated with a lesion image is set. The details of processing of S409 will be described later with reference to FIGS. 5A to 5C and FIGS. 6A and 6B.

In S410, the system control unit 50 judges whether the user has selected the photographer information frame 303 on the LV screen. The selection of the photographer information frame 303 is made by touching the photographer information frame 303, or by moving the selection frame to the photographer information frame 303 through a dial operation or a direction key operation and depressing the SET button 75. In a case where the photographer information frame 303 has been selected, processing proceeds to S411; otherwise, processing proceeds to S412.

In S411, the system control unit 50 executes processing in the photographer information obtainment mode. Through this processing, medical information (here, photographer information) to be associated with a lesion image is set. The details of processing of S411 will be described later with reference to FIGS. 5A to 5C and FIGS. 6A and 6B.

In S412, the system control unit 50 judges whether the user has selected the area information frame 307 on the LV screen. The selection of the area information frame 307 is made by touching the area information frame 307, or by moving the selection frame to the area information frame 307 through a dial operation or a direction key operation and depressing the SET button 75. In a case where the area information frame 307 has been selected, processing proceeds to S413; otherwise, processing proceeds to S414.

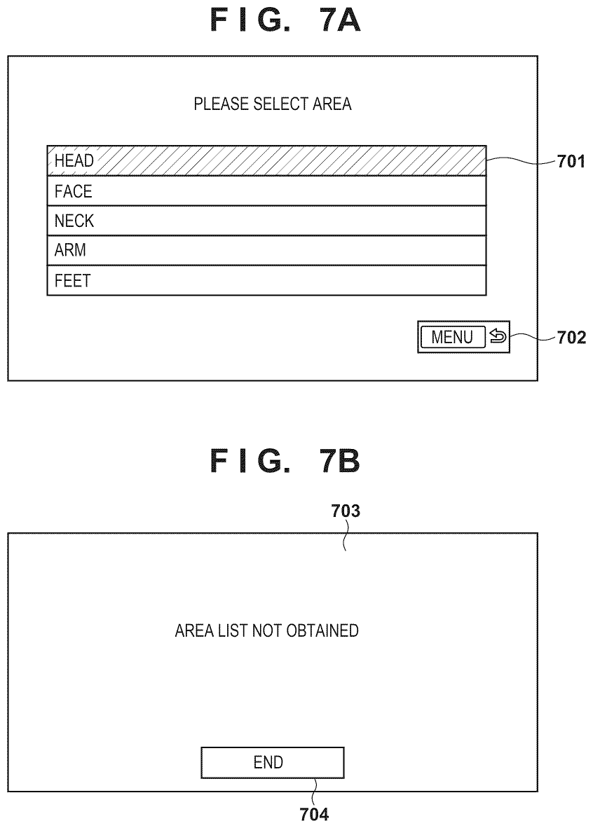

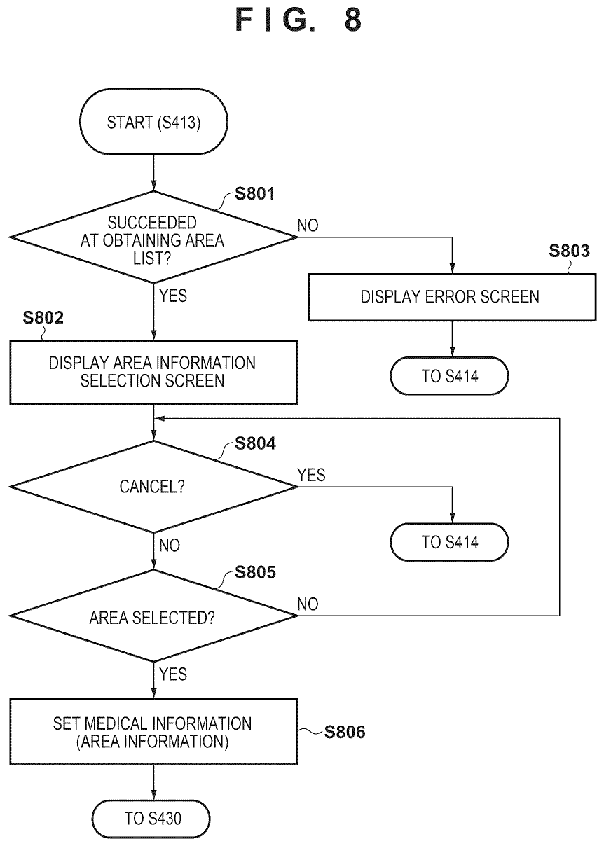

In S413, the system control unit 50 executes processing in the area information obtainment mode. Through this processing, medical information (here, area information) to be associated with a lesion image is set. The details of processing of S413 will be described later with reference to FIGS. 7A and 7B and FIG. 8.

With the completion of setting of the medical information through processing of S409, S411, or S413, the system control unit 50 turns OFF the power saving flag in S430. Therefore, according to processing in the medical mode of the present embodiment (FIGS. 4A to 4D), a power saving function is disabled upon obtainment and setting of at least a part of the medical information.

In S414, the system control unit 50 determines whether the user has depressed the display switching button 83. In a case where the display switching button 83 has been depressed, processing proceeds to S415; otherwise, processing proceeds to S418.

In S415, the system control unit 50 determines whether the LV display setting is the medical information display. In a case where the LV display setting is the medical information display, processing proceeds to S416; otherwise, processing proceeds to S417.

In S416, the system control unit 50 sets (changes) the LV display setting to the shooting parameter display.

In S417, the system control unit 50 sets (changes) the LV display setting to the medical information display.

In S418, the system control unit 50 determines whether the user has performed a dial operation. In a case where the user has performed a dial operation, processing proceeds to $419; otherwise, processing proceeds to S422.

In S419, the system control unit 50 determines whether the LV display setting is the medical information display. In a case where the LV display setting is the medical information display, processing proceeds to S420; otherwise, processing proceeds to S421.

In S420, the system control unit 50 moves the selection frame for the medical information on the LV screen corresponding to the medical information display (FIG. 3A). Note that in a case where the dial operation has been performed during half-pressing of the shutter button 61, the system control unit 50 may disable the movement of the selection frame for the medical information.

In S421, the system control unit 50 changes a shooting parameter corresponding to the operated dial. The digital camera 100 has, for example, a shutter speed, an f-number, exposure correction, and ISO as shooting parameters. The shooting parameters are assigned on a per-dial basis, and this assignment may vary depending on a shooting mode.

In S422, the system control unit 50 determines whether the user has half-pressed the shutter button 61. In a case where the user has half-pressed the shutter button 61, processing proceeds to S423; otherwise, processing proceeds to S427.

In S423, the system control unit 50 sets the LV display setting to the medical information display.

In S424, the system control unit 50 displays the LV screen corresponding to the LV display setting on the display unit 28. As the LV display setting has been set to the medical information display in S423, the LV screen corresponding to the medical information display (FIG. 3A) is displayed in S424.

In S425, the system control unit 50 determines whether the user has full-pressed the shutter button. In a case where the user has full-pressed the shutter button, processing proceeds to S426; otherwise, processing returns to S422.

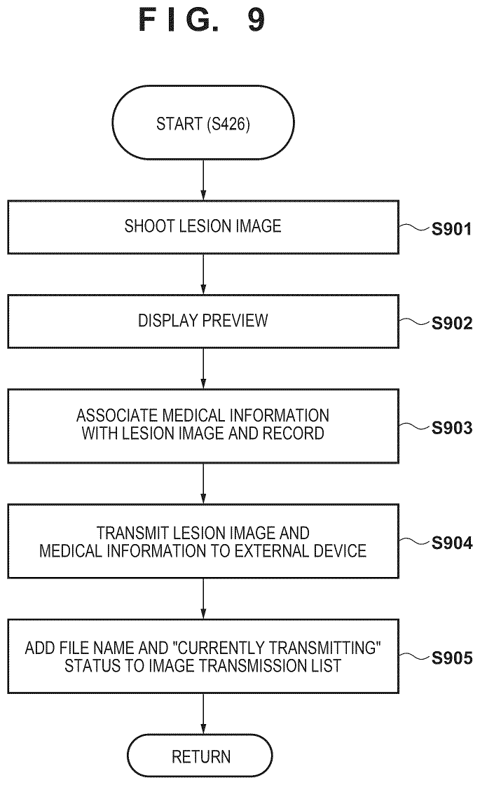

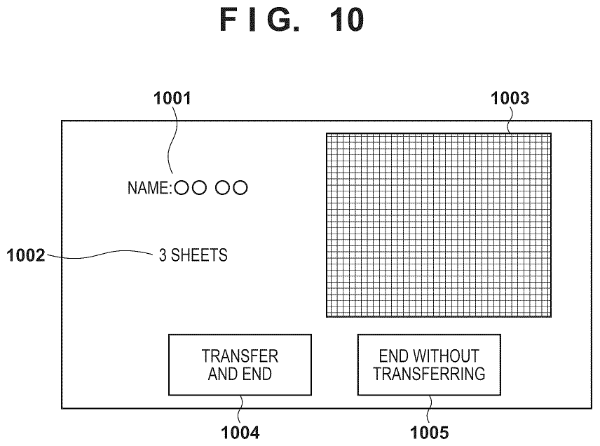

In S426, the system control unit 50 executes processing in the lesion shooting mode. The details of processing of S426 will be described later with reference to FIG. 9.

In S427, the system control unit 50 judges whether the user has selected the end button 308 on the LV screen. The selection of the end button 308 is made by touching the end button 308, or by moving the selection frame to the end button 308 through a dial operation or a direction key operation and depressing the SET button 75. Alternatively, as another example, the system control unit 50 may determine that the end button 308 has been selected in a case where an end button function is assigned to a button included in other operation members 70b and the pertinent button has been pressed. In a case where the end button 308 has been selected, processing proceeds to S428; otherwise, processing proceeds to S431.

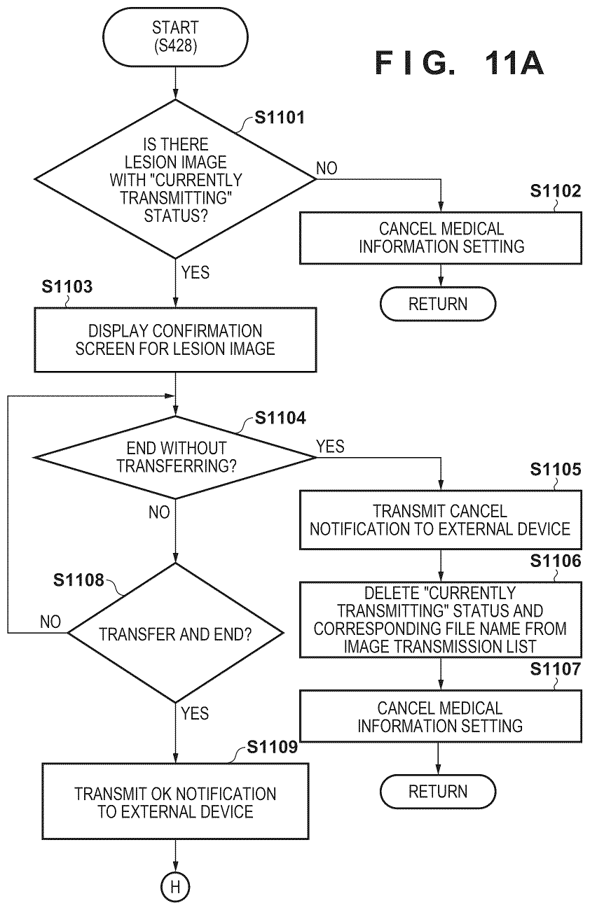

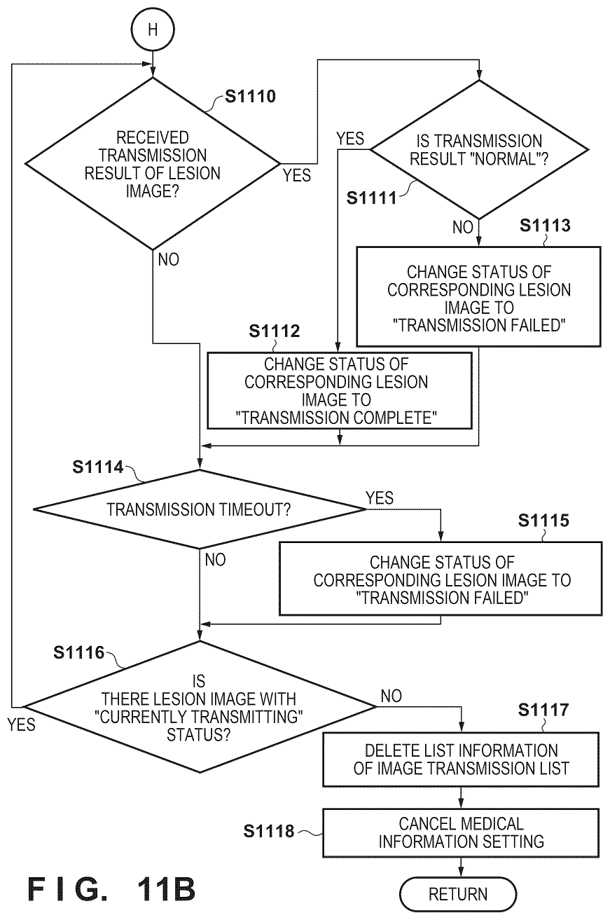

In S428, the system control unit 50 executes processing in the lesion shooting end mode. As a result of this processing, the setting of the medical information that has been set to be associated with the lesion image is cancelled. The setting of the medical information is cancelled by, for example, clearing (deleting) the medical information held in the system memory 52. The details of processing of S428 will be described later with reference to FIG. 10 and FIGS. 11A and 11B.

In S429, the system control unit 50 turns ON the power saving flag. Therefore, according to processing in the medical mode of the present embodiment (FIGS. 4A to 4D), the power saving function is enabled upon cancellation of the setting of the medical information.

In S431, the system control unit 50 judges whether the medical mode is to be ended. For example, it is determined that the medical mode is to be ended in a case where the user has switched the medical mode to OFF, or the user has instructed to turn OFF the power source of the digital camera 100 by operating the power source switch 72. In a case where the medical mode is to be ended, processing proceeds to S432; otherwise, processing returns to S406.

In S432, the system control unit 50 cancels the setting of the medical information. As stated earlier, the setting of the medical information is cancelled by, for example, clearing (deleting) the medical information held in the system memory 52.

In S433, the system control unit 50 turns ON the power saving flag. Therefore, according to processing in the medical mode of the present embodiment (FIGS. 4A to 4D), the setting of the medical information is cancelled and the power saving function is enabled upon ending the medical mode (switching the medical mode from ON to OFF).

In S436, the system control unit 50 determines whether the power saving flag is ON. In a case where the power saving flag is ON, processing proceeds to S437; otherwise, processing proceeds to S438.

In S437, the system control unit 50 executes power saving processing (processing for causing the digital camera 100 to transition to a power saving state). For example, the system control unit 50 executes processing for turning OFF the power source of the digital camera 100 as the power saving processing. Alternatively, the power saving processing may be one of the processes indicated below, or a combination of two or more processes included among the processes indicated below.
Turning OFF the display unit 28
Reducing the luminance of the display unit 28
Reducing the LV display rate of the display unit 28
Reducing the LV display resolution of the display unit 28
Stopping a communication operation of the communication unit 54
Turning OFF the image capturing unit 22
Turning OFF the EVF 29

Note that in a case where the display unit 28 has been turned OFF or reduced in luminance through the power saving processing, there is a possibility that the user mistakenly considers that the power source of the digital camera 100 has been turned OFF, and turns OFF the power source of the digital camera 100 by operating the power source switch 72. To avoid the occurrence of this situation, the system control unit 50 may disable an operation on the power source switch 72.

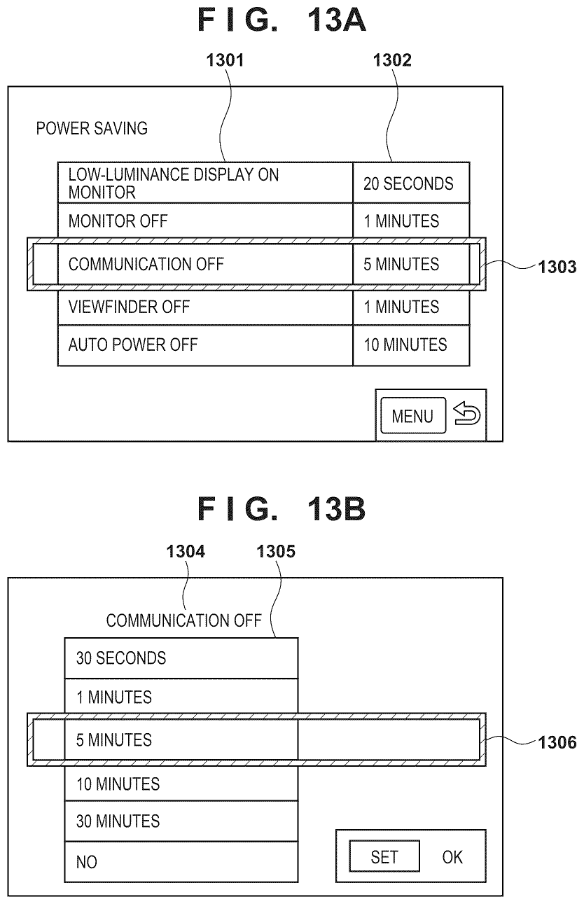

Furthermore, the digital camera 100 may be configured so that the user can set the contents of the power saving processing. A setting screen for making this setting will be described later with reference to FIGS. 13A and 13B.

In S438, the system control unit 50 provides a notification that prompts the user to select whether to end shooting. This notification is provided by, for example, displaying a message that inquires of the user whether to end shooting (an end-of-shooting confirmation message).

In S439, the system control unit 50 determines whether the user has instructed (selected) to end shooting. For example, in S438, an “end shooting” button and a “continue shooting” button are displayed together with the end-of-shooting confirmation message; in a case where the user has selected the “end shooting” button, the system control unit 50 determines that the user has instructed (selected) to end shooting. In a case where the user has selected the “continue shooting” button, the system control unit 50 determines that the user has not instructed (selected) to end shooting. In a case where the user has instructed to end shooting, processing proceeds to S428; otherwise, processing returns to S406.

Note that the external system that communicates with the digital camera 100 may include an electronic medical record terminal, although the illustration thereof is omitted. In this case, the system control unit 50 may detect that a display target patient for whom an electronic medical record is to be displayed on the electronic medical record terminal has been changed from a current patient (a first patient) to another patient (a second patient) by communicating with the external system. In response to the detection of the change in the display target patient, the system control unit 50 may cancel the setting of the medical information and turn ON the power saving flag.

Furthermore, the system control unit 50 may detect the end of hours of medical practice in a medical facility that uses the digital camera 100 by communicating with the external system. For example, the digital camera 100 can obtain medical practice schedule information, which includes the ending time of hours of medical practice, from the external device, and detect the end of hours of medical practice by judging whether the current time is past the ending time. In a case where the end of hours of medical practice has been detected, the system control unit 50 may cancel the setting of the medical information and turn ON the power saving flag by executing processing in the lesion shooting end mode.

Furthermore, in S429 and S433, the system control unit 50 may judge whether a time period to the starting time of the medical practice for the next patient is shorter than a threshold by communicating with the external system. For example, the system control unit 50 can obtain medical practice schedule information from the external device, and judge whether the time period to the starting time of the medical practice for the next patient is shorter than the threshold on the basis of the current time and the medical practice schedule information. In a case were the time period to the starting time of the medical practice for the next patient is shorter than the threshold, the system control unit 50 may keep the power saving flag OFF, without turning ON the same. That is to say, even in a case where the medical information has not been set (a case where the setting of the medical information has been cancelled), if the time period to the starting time of the medical practice for the next patient is shorter than the threshold, the system control unit 50 may perform control so as not to cause the digital camera 100 to transition to the power saving state, even when the unoperated state has continued for the predetermined time period.

### Processing in Patient Information Obtainment Mode and Photographer Information Obtainment Mode

Processing in the patient information obtainment mode and the photographer information obtainment mode will be described with reference to FIGS. 5A to 5C and FIGS. 6A and 6B. The following description, which will be provided with reference to FIGS. 5A to 5C and FIGS. 6A and 6B, applies to both of processing in the patient information obtainment mode and processing in the photographer information obtainment mode, unless specifically stated otherwise.

FIG. 5A is a diagram showing an example of a barcode obtainment screen displayed in the patient information obtainment mode and the photographer information obtainment mode.

501 indicates a display region of an LV image of a subject. In the display region 501, in order to emphasize a region 511 in which a barcode should be located for shooting, a portion other than the region 511 is displayed in a dark state.

502 is a MENU button. In a case where the user wishes to exit the barcode obtainment screen (to end the patient information obtainment mode or the photographer information obtainment mode), the user selects the MENU button 502.

512 is a message that prompts the user to shoot the barcode. Note that the message 512 may include information indicating whether the current mode is the patient information obtainment mode or the photographer information obtainment mode. For example, in the case of the patient information obtainment mode, the message 512 may be “please shoot the barcode of the patient”, whereas in the case of the photographer information obtainment mode, the message 512 may be “please shoot the barcode of the photographer”.

FIG. 5B is a diagram showing an example of a confirmation screen of patient information (in the case of the patient information obtainment mode) or photographer information (in the case of the photographer information obtainment mode) corresponding to the shot barcode. The patient information and the photographer information are received from an external device. 503 is a region that displays the patient information or the photographer information received from the external device. In the examples of FIGS. 5A to 5C, a full name of a patient or a full name of a photographer included in the received patient information or photographer information is displayed.

504 is an OK button. In a case where information displayed in the region 503 is correct, the user selects the OK button 504.

505 is a re-shoot button 505. In a case where the user wishes to re-shoot the barcode, the user selects the re-shoot button 505.

FIG. 5C is a diagram showing an example of an error screen displayed in a case where the obtainment (reception) of the patient information or the photographer information has failed.

506 is a message announcing an error. The displayed message may be changed in accordance with the content of the error.

507 is an end button. In a case where the user is to give up the obtainment of the patient information or the photographer information, the user selects the end button 507.

508 is a re-shoot button 508. In a case where the user wishes to re-shoot the barcode in an attempt to obtain the patient information or the photographer information again, the user selects the re-shoot button 508.

FIGS. 6A and 6B are flowcharts of processing in the patient information obtainment mode and the photographer information obtainment mode.

In S601, the system control unit 50 displays the barcode obtainment screen shown in FIG. 5A on the display unit 28.

In S602, the system control unit 50 judges whether a cancel instruction has been issued. The system control unit 50 determines that the cancel instruction has been issued in a case where the MENU button 502 has been selected on the barcode obtainment screen, or in a case where the menu button 81 has been depressed. In a case where the cancel instruction has been issued, the patient information obtainment mode or the photographer information obtainment mode is ended, and processing proceeds to S410 (in the case of the patient information obtainment mode) or S412 (in the case of the photographer information obtainment mode). In a case where the cancel instruction has not been issued, processing proceeds to S603.

In S603, the system control unit 50 determines whether the shutter button 61 has been full-pressed. In a case where the shutter button 61 has been full-pressed, processing proceeds to S604; otherwise, processing returns to S602.

In S604, the system control unit 50 shoots a barcode, and transmits an obtained barcode image to an external device via the communication unit 54. Alternatively, the system control unit 50 may obtain a code by analyzing the barcode image, and transmit obtained code information to the external device via the communication unit 54.

In S605, the system control unit 50 judges whether a response has been received from the external device. The system control unit 50 repeats the judgment of S605 until the response is received; once the response has been received, processing proceeds to 606.

In S606, the system control unit 50 determines whether the received response is normal. The system control unit 50 determines that the response is abnormal in a case where medical information (patient information or photographer information) in the response is corrupted or the response does not include intended medical information. In a case where the response is normal, processing proceeds to S607; otherwise, processing proceeds to S608.

In S607, based on the patient information or the photographer information included in the received response, the system control unit 50 displays the confirmation screen shown in FIG. 5B on the display unit 28.

In S608, the system control unit 50 displays an error screen shown in FIG. 5C on the display unit 28.

In S609, the system control unit 50 judges whether an end instruction has been issued. The system control unit 50 determines that the end instruction has been issued in a case where the user has selected the end button 507 on the error screen, or in a case where the user has moved a selection frame to the end button 507 through a dial operation or a direction key operation and depressed the SET button 75. In a case where the end instruction has been issued, the patient information obtainment mode or the photographer information obtainment mode is ended, and processing proceeds to S410 (in the case of the patient information obtainment mode) or S412 (in the case of the photographer information obtainment mode). In a case where the end instruction has not been issued, processing proceeds to S610.

In S610, the system control unit 50 judges whether a re-shoot instruction has been issued. The system control unit 50 determines that the re-shoot instruction has been issued in a case where the user has selected the re-shoot button 508 on the error screen, or in a case where the user has moved the selection frame to the re-shoot button 508 through a dial operation or a direction key operation and depressed the SET button 75. In a case where the re-shoot instruction has been issued, processing returns to S601; otherwise, processing returns to S609.

In S611, the system control unit 50 judges whether the user has issued an OK instruction. The system control unit 50 determines that the OK instruction has been issued in a case where the user has selected the OK button 504 on the confirmation screen, or in a case where the user has moved the selection frame to the OK button 504 through a dial operation or a direction key operation and depressed the SET button 75. In a case where the OK instruction has been issued, processing proceeds to S613; otherwise, processing proceeds to S612.

In S612, the system control unit 50 judges whether a re-shoot instruction has been issued. The system control unit 50 determines that the re-shoot instruction has been issued in a case where the user has selected the re-shoot button 505 on the confirmation screen, or in a case where the user has moved the selection frame to the re-shoot button 505 through a dial operation or a direction key operation and depressed the SET button 75. In a case where the re-shoot instruction has been issued, processing returns to S601; otherwise, processing returns to S611.

In S613, the system control unit 50 holds (stores) the medical information (patient information in the case of the patient information obtainment mode, and photographer information in the case of the photographer information obtainment mode) included in the response received in S605 in the system memory 52. As a result, the medical information (patient information or photographer information) to be associated with a lesion image is set. Furthermore, in a case where the LV screen of FIG. 3A is displayed thereafter, the full name of the photographer is displayed in the photographer information frame 303 (in the case of the photographer information obtainment mode), and the full name of the patient is displayed in the patient information frame 305 (in the case of the patient information obtainment mode).

### Processing in Area Information Obtainment Mode

Processing in the area information obtainment mode will be described with reference to FIGS. 7A and 7B and FIG. 8.

FIG. 7A is a diagram showing an example of an area information selection screen. 701 is an area list obtained in S404. 702 is a MENU button. In a case where the user wishes to exit the area information selection screen (to end the area information obtainment mode), the user selects the MENU button 702. FIG. 7B is a diagram showing an example of an error screen. 703 is a message announcing an error. The displayed message may be changed in accordance with the content of the error. 704 is an end button. When the user has selected the end button 704, the area information obtainment mode is ended.

FIG. 8 is a flowchart of processing in the area information obtainment mode. In S801, the system control unit 50 determines whether it has been able to obtain an area list from an external device in S404. In a case where it has been able to obtain the area list, processing proceeds to S802; otherwise, processing proceeds to S803.

In S802, the system control unit 50 displays the area information selection screen shown in FIG. 7A on the display unit 28.

In S803, the system control unit 50 displays the error screen shown in FIG. 7B on the display unit 28. When the end button 704 has been selected on the error screen, processing proceeds to S414.

In S804, the system control unit 50 judges whether a cancel instruction has been issued. The system control unit 50 determines that the cancel instruction has been issued in a case where the MENU button 702 has been selected on the area information selection screen, or in a case where the menu button 81 has been depressed. In a case where the cancel instruction has been issued, the area information obtainment mode is ended, and processing proceeds to S414. In a case where the cancel instruction has not been issued, processing proceeds to S805.

In S805, the system control unit 50 judges whether an area has been selected from the area list 701. The user can select an area by touching one of the plurality of areas included in the area list. Alternatively, the user can select an area by moving a selection frame to the position of one of the plurality of areas included in the area list through a dial operation or a direction key operation and depressing the SET button 75. In a case where an area has been selected, processing proceeds to S806; otherwise, processing returns to S804.

In S806, the system control unit 50 holds (stores) area information indicating the area selected in S805 in the system memory 52. As a result, the area information to be associated with a lesion image is set. Also, in a case where the LV screen of FIG. 3A is displayed thereafter, the area information is displayed in the area information frame 307.

### Processing in Lesion Shooting Mode

FIG. 9 is a flowchart of processing in the lesion shooting mode. In S901, the system control unit 50 shoots a lesion image and saves the same in the memory 32.

In S902, the system control unit 50 preview-displays the lesion image on the display unit 28. As one example, the system control unit 50 may display the set medical information (at least one of the patient information, photographer information, and area information) in such a manner that the set medical information is overlaid on the lesion image that has been preview-displayed. Furthermore, execution and inexecution of the preview display may be switchable via a user setting. Furthermore, a display time period of the preview display may be changeable via a user setting.

In S903, the system control unit 50 associates the set medical information (at least one of the patient information, photographer information, and area information) with the lesion image, and records the lesion image in the recording medium 200. Although a method of association is not limited in particular, for example, the system control unit 50 can write the medical information into a metadata region or the like of an image file including the lesion image. Alternatively, the system control unit 50 may generate a metadata file including the medical information, and associate an image file including the lesion image with the metadata file.

In S904, the system control unit 50 transmits the lesion image and the medical information associated with the lesion image to an external device via the communication unit 54.

In S905, the system control unit 50 adds information that identifies the lesion image (e.g., the file name of the image file), as well as a “currently transmitting” status indicating that this lesion image is currently being transmitted, to an image transmission list held in the memory 32.

### Processing in Lesion Shooting End Mode

Processing in the lesion shooting end mode will be described with reference to FIG. 10 and FIGS. 11A and 11B.

FIG. 10 is a diagram showing an example of a confirmation screen for a lesion image. On the confirmation screen, the user can confirm the lesion image shot in S901 and the medical information (in the example of FIG. 10, the full name of the patient included in the patient information) associated with this lesion image in S903.

1001 is a display region of medical information (in the example of FIG. 10, the full name of the patient included in the patient information) associated with a lesion image with a “currently transmitting” status in the image transmission list. In a case where there are a plurality of lesion images with the “currently transmitting” status, the display region 1001 may display medical information of the lesion image that was shot first or last, or may display pieces of medical information of all lesion images.

1002 is a display region that displays the number of lesion images with the “currently transmitting” status in the image transmission list.

1003 is a display region that displays a thumbnail image of a lesion image with the “currently transmitting” status in the image transmission list. In a case where there are a plurality of lesion images with the “currently transmitting” status, the display region 1003 may display a thumbnail image of the lesion image that was shot first or last, or may display thumbnail images of all lesion images.

1004 is a button for causing the user to select to transfer the lesion image to the medical system and end lesion shooting.

1005 is a button for causing the user to select to end lesion shooting without transferring the lesion image to the medical system.

FIGS. 11A and 11B are flowcharts of processing in the lesion shooting end mode. In S1101, the system control unit 50 determines whether there is a lesion image with the “currently transmitting” status in the image transmission list. In a case where there is a lesion image with the “currently transmitting” status, processing proceeds to S1103; otherwise, processing proceeds to S1102.

In S1102, the system control unit 50 clears the medical information (patient information, photographer information, and area information) held in the system memory 52. As a result, the setting of the medical information is cancelled. Thereafter, the lesion shooting end mode is ended, and processing proceeds to S429.

In S1103, the system control unit 50 displays the confirmation screen for the lesion image shown in FIG. 10.

In S1104, the system control unit 50 judges whether an instruction for ending lesion shooting without transferring the lesion image has been issued. The system control unit 50 determines that the instruction for ending lesion shooting without transferring the lesion image has been issued in a case where the user has selected the “end without transfer” button 1005 on the confirmation screen, or in a case where the user has moved a selection frame to the “end without transfer” button 1005 through a dial operation or a direction key operation and depressed the SET button 75. In a case where the instruction for ending lesion shooting without transferring the lesion image has been issued, processing proceeds to S1105; otherwise, processing proceeds to S1108.

In S1105, the system control unit 50 transmits a cancel notification to an external device via the communication unit 54.

In S1106, the system control unit 50 deletes the “currently transmitting” status and the file name of the corresponding lesion image from the image transmission list.

In S1107, the system control unit 50 clears the medical information (patient information, photographer information, and area information) held in the system memory 52. As a result, the setting of the medical information is cancelled. Thereafter, the lesion shooting end mode is ended, and processing proceeds to $429.

In S1108, the system control unit 50 judges whether an instruction for transferring the lesion image and ending lesion shooting has been issued. The system control unit 50 determines that the instruction for transferring the lesion image and ending lesion shooting has been issued in a case where the user has selected the “transfer and end” button 1004 on the confirmation screen, or in a case where the user has moved a selection frame to the “transfer and end” button 1004 through a dial operation or a direction key operation and depressed the SET button 75. In a case where the instruction for transferring the lesion image and ending lesion shooting has been issued, processing proceeds to S1109; otherwise, processing returns to S1104.

In S1109, the system control unit 50 transmits an OK notification to the external device via the communication unit 54.

In S1110, the system control unit 50 determines whether a transmission result of the lesion image transmitted in S904 has already been received from the external device. In a case where the transmission result has already been received, processing proceeds to S1111; otherwise, processing proceeds to S1114.

In S1111, the system control unit 50 determines whether the transmission result received from the external device is normal. In a case where the transmission result is normal, processing proceeds to S1112; otherwise, processing proceeds to S1113.

In S1112, the system control unit 50 changes the status of the lesion image corresponding to the received transmission result to “transmission complete” in the image transmission list.

In S1113, the system control unit 50 changes the status of the lesion image corresponding to the received transmission result to “transmission failed” in the image transmission list.

In S1114, the system control unit 50 determines whether the transmission result has been received from the external device within a certain time period since the start of transmission of the lesion image to the external device in S904 (i.e., whether a transmission timeout has occurred). In a case where the transmission result has not been received from the external device within the certain time period (in a case where the transmission timeout has occurred), processing proceeds to S1115; otherwise, processing proceeds to S1116.

In S1115, the system control unit 50 changes the status of the lesion image for which the transmission timeout has occurred to “transmission failed” in the image transmission list.

In S1116, the system control unit 50 determines whether there is a lesion image with the “currently transmitting” status in the image transmission list. In a case where there is a lesion image with the “currently transmitting” status, processing returns to S1110; otherwise, processing proceeds to S1117.

In S1117, the system control unit 50 deletes list information (the file name and the status of each lesion image) included in the image transmission list.

In S1118, the system control unit 50 clears the held medical information (patient information, photographer information, and area information). As a result, the setting of the medical information is cancelled. Thereafter, the lesion shooting end mode is ended, and processing proceeds to S429.

### ON/OFF Switching Processing for Medical Mode

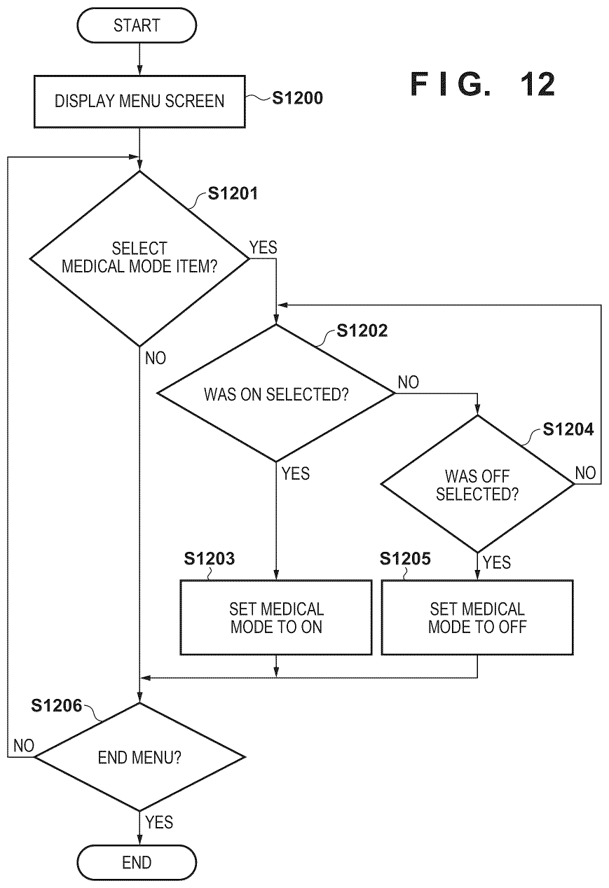

FIG. 12 is a flowchart of processing for switching between ON and OFF of the medical mode. Processing of the present flowchart is started in a case where the user has depressed the menu button 81, regardless of whether the medical mode is ON or OFF. Note that while the system control unit 50 is executing processing of the patient information obtainment mode, the photographer information obtainment mode, the area information obtainment mode, the lesion shooting mode, or the lesion shooting end mode, processing of the present flowchart is not started even if the menu button 81 has been depressed. Furthermore, the system control unit 50 may perform control so that, in a case where the power saving flag is OFF, processing of the present flowchart is not started even if the menu button 81 has been depressed. Processing of each step in the present flowchart is realized by the system control unit 50 deploying the program stored in the nonvolatile memory 56 to the system memory 52 and executing the program, unless specifically stated otherwise.

In S1200, the system control unit 50 displays a menu screen on the display unit 28.

In S1201, the system control unit 50 judges whether the user has selected an item of the medical mode on the menu screen. The user can select an item of the medical mode by touching the item of the medical mode on the menu screen. Alternatively, the user can select an item of the medical mode by moving a selection frame to the position of the item of the medical mode through a dial operation or a direction key operation and depressing the SET button 75. In a case where an item of the medical mode has been selected, the system control unit 50 displays ON and OFF as a sub item of the item of the medical mode, and processing proceeds to S1202. In a case where an item of the medical mode has not been selected, processing proceeds to S1206.

In S1202, the system control unit 50 judges whether the user has selected ON in the sub item of the item of the medical mode. The user can select ON by touching ON in the sub item. Alternatively, the user can select ON by moving a selection frame to the position of ON through a dial operation or a direction key operation and depressing the SET button 75. In a case where ON has been selected, processing proceeds to $1203; otherwise, processing proceeds to S1204.

In S1203, the system control unit 50 sets the medical mode to ON (e.g., changes the operation mode from the medical mode to a normal mode).

In S1204, the system control unit 50 judges whether the user has selected OFF in the sub item of the item of the medical mode. The user can select OFF by touching OFF in the sub item. Alternatively, the user can select OFF by moving a selection frame to the position of OFF through a dial operation or a direction key operation and depressing the SET button 75. In a case where OFF has been selected, processing proceeds to S1205; otherwise, processing returns to S1202.

In S1205, the system control unit 50 sets the medical mode to OFF. Once the medical mode has been set to OFF, for example, the operation mode is changed from the medical mode to the normal mode.

In S1206, the system control unit 50 judges whether an instruction for ending a menu has been issued. For example, the system control unit 50 determines that the instruction for ending the menu has been issued in a case where the user has touched a menu end button on the menu screen. In a case where the instruction for ending the menu has been issued, processing of the present flowchart is ended. In a case where the instruction for ending the menu has not been issued, processing returns to S1201.

### Examples of Setting Screens of Power Saving Processing

Examples of setting screens of the power saving processing according to the present embodiment will be described with reference to FIGS. 13A and 13B. The system control unit 50 can display, as the setting screens, a list display screen of power saving items and a detailed setting screen of power saving items.

FIG. 13A is a diagram showing an example of the list display screen of power saving items. 1301 is a display region of the power saving items. “Low-luminance display on monitor” corresponds to processing for “reducing the luminance of the display unit 28” described in S437. Also, in order to improve the power saving performance, “low-luminance display on monitor” may include processing for “reducing the LV display rate of the display unit 28” and processing for “reducing the LV display resolution of the display unit 28” described in S437. Alternatively, the display region 1301 may include individual items in connection with the processing for “reducing the LV display rate of the display unit 28” and the processing for “reducing the LV display resolution of the display unit 28”, respectively.

“Monitor OFF” corresponds to processing for “turning OFF the display unit 28” described in S437. Also, in order to improve the power saving performance, “monitor OFF” may include processing for “turning OFF the image capturing unit 22” described in S437. Alternatively, the display region 1301 may include an individual item corresponding to the processing for “turning OFF the image capturing unit 22”.

“Communication OFF” corresponds to processing for stopping a communication operation of the communication unit 54″ described in S437. For example, the system control unit 50 can stop the communication operation of the communication unit 54 by stopping a power supply to the communication unit 54. “Viewfinder OFF” corresponds to processing for “turning OFF the EVF 29” described in S437.

“Auto power OFF” corresponds to processing for turning OFF the power of the entire system of the digital camera 100 (processing for “turning OFF the power source of the digital camera 100” described in S437).

1302 is a display region that displays a set time period of each power saving item. As shown in FIG. 13A, the user can set different time periods for different power saving items. Therefore, the judgment of S407 is made for each power saving item. Also, in making the judgment for each power saving item, the time periods that have been set for the respective power saving items are used as the “predetermined time period”.

1303 is a selection frame indicating which item is selected from among the plurality of power saving items that are displayed. The user can move the selection frame 1303 through a dial operation or a direction key operation, and select any power saving item by depressing the SET button 75.

FIG. 13B is a diagram showing an example of a detailed setting screen of the power saving item corresponding to “communication OFF”. When “communication OFF” has been selected on the list display screen of FIG. 13A, the detailed setting screen of FIG. 13B is displayed.

1304 is a display region that displays the power saving item corresponding to the detailed setting screen that is displayed.

1305 is a display region that displays a list of settable values (time periods).

1306 is a selection frame indicating which value is selected from among the plurality of settable values (time periods) that are displayed. The user can move the selection frame 1306 through a dial operation or a direction key operation, and select any value by depressing the SET button 75. When the user has selected a specific value, the selected value is set as a time period of “communication OFF”.

Note that the system control unit 50 may perform control to prohibit changing of settings of the power saving processing in a case the power saving flag is OFF (or in a case where medical information is held in the system memory 52). In this case, changing of the “predetermined time period” used in S407 is prohibited.

### Summary of First Embodiment

As described above, according to the first embodiment, the digital camera 100 sets specific information (e.g., medical information). The digital camera 100 associates the set specific information with an image shot using the image capturing unit 22 (e.g., a lesion image, which is one example of a medical image). In a case where the specific information has not been set, the digital camera 100 performs control so that the digital camera 100 transitions to the power saving state when the unoperated state has continued for a predetermined time period. Also, in a case where the specific information has been set, the digital camera 100 performs control so that the digital camera 100 does not transition to the power saving state even if the unoperated state has continued for the predetermined time period.

In a state where specific information to be associated with a shot image has been set, it is considered that the possibility of the user performing shooting in a short while is relatively high. In this state, the digital camera 100 performs control so that the digital camera 100 does not transition to the power saving state even if the unoperated state has continued for the predetermined time period. Therefore, the present embodiment reduces the possibility that the digital camera 100 transitions to the power saving state against the user's intention.

Conversely, in a state where specific information to be associated with a shot image has not been set, it is considered that the possibility of the user performing shooting in a short while is relatively low. In this state, the digital camera 100 performs control so that the digital camera 100 transitions to the power saving state when the unoperated state has continued for the predetermined time period. Therefore, the present embodiment suppresses an increase in a wasteful power consumption.

As described above, the present embodiment can reduce the possibility that an image capturing apparatus transitions to the power saving state against the user's intention while suppressing an increase in a wasteful power consumption.

Note that each type of processing that has been described above as being executed by the system control unit 50 may be executed by one item of hardware, or may be executed by a plurality of items of hardware (e.g., a plurality of processors and circuits) sharing the processing.

Also, although the present embodiment has been described using an exemplary case where the digital camera 100 is used as an image capturing apparatus, any device can be used as an image capturing apparatus of the present embodiment as long as it is a device including image capturing means. That is to say, the present embodiment is applicable to a mobile terminal like a smartphone, a personal computer, a PDA, a mobile telephone terminal, a mobile image viewer, a display-equipped printer apparatus, a digital photo frame, a music player, a game device, an electronic book reader, and so forth.

Furthermore, the present embodiment is applicable not only to a body of an image capturing apparatus, but also to a control apparatus that communicates with the image capturing apparatus (including a network camera) via wired or wireless communication and remotely controls the image capturing apparatus. Examples of an apparatus that remotely controls an image capturing apparatus are such apparatuses as a smartphone, a tablet PC, and a desktop PC. An image capturing apparatus can be remotely controlled by providing, from the control apparatus side, a notification of a command for causing the image capturing apparatus to perform various types of operations and settings on the basis of an operation that has been performed on the control apparatus side and processing that has been executed on the control apparatus side. Furthermore, the control apparatus side may be capable of receiving a live-view image shot by the image capturing apparatus via wired or wireless communication and displaying the live-view image.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

1. An image capturing apparatus including an image capturing unit, comprising:
a switching unit configured to switch between ON and OFF of a medical mode of the image capturing apparatus, wherein the medical mode is an operation mode for shooting a medical image;
a setting unit configured to set specific information;
an association unit configured to associate the set specific information with an image that has been shot using the image capturing unit; and
a control unit configured to perform, in a case where the medical mode is ON, control to
in a case where the specific information has not been set, cause the image capturing apparatus to transition to a power saving state when a state where a user has not operated the image capturing apparatus has continued for a predetermined time period, and
in a case where the specific information has been set, not cause the image capturing apparatus to transition to the power saving state even if the state where the user has not operated the image capturing apparatus has continued for the predetermined time period.

2. The image capturing apparatus according to claim 1, wherein
the image is a medical image of a first patient, and
the specific information includes at least one of information related to the first patient, information related to a photographer, and information related to a shooting target area of the first patient.

3. The image capturing apparatus according to claim 2, further comprising:
a communication unit; and
a reception unit configured to receive the specific information from an external system via the communication unit.

4. The image capturing apparatus according to claim 3, further comprising:
a transmission unit configured to transmit, to the external system, the image with which the set specific information has been associated.

5. The image capturing apparatus according to claim 3, further comprising:
a first detection unit configured to detect, by communicating with the external system via the communication unit, that a display target patient for whom an electronic medical record is to be displayed on an electronic medical record terminal included in the external system has been changed from the first patient to a second patient,
wherein the setting unit cancels the setting of the specific information in response to detection of the change in the display target patient from the first patient to the second patient.

6. The image capturing apparatus according to claim 3, further comprising:
a second detection unit configured to detect, by communicating with the external system via the communication unit, an end of hours of medical practice in a medical facility that uses the image capturing apparatus,
wherein the setting unit cancels the setting of the specific information in response to detection of the end of hours of medical practice.

7. The image capturing apparatus according to claim 3, further comprising:
a judgment unit configured to judge, by communicating with the external system via the communication unit, whether a time period to a starting time of medical practice for a next patient is shorter than a threshold,
wherein even in a case where the specific information has not been set, if the time period to the starting time of the medical practice for the next patient is shorter than the threshold, the control unit performs control so as not to cause the image capturing apparatus to transition to the power saving state even if the state where the user has not operated the image capturing apparatus has continued for the predetermined time period.

8. The image capturing apparatus according to claim 3, wherein
causing the image capturing apparatus to transition to the power saving state includes stopping a communication operation of the communication unit.

9. The image capturing apparatus according to claim 1, wherein
the setting unit cancels the setting of the specific information when the user has issued an instruction for ending shooting.

10. The image capturing apparatus according to claim 1, wherein:
the setting unit sets the specific information in a case where the predetermined operation mode is ON, and cancels the setting of the specific information when the predetermined operation mode has been switched from ON to OFF.

11. The image capturing apparatus according to claim 1, wherein
the setting unit cancels the setting of the specific information when the user has issued an instruction for turning OFF a power source of the image capturing apparatus.

12. The image capturing apparatus according to claim 1, wherein
in a case where the specific information has been set, the control unit provides a notification that prompts the user to select whether to end shooting when the state where the user has not operated the image capturing apparatus has continued for the predetermined time period, and
the setting unit cancels the setting of the specific information in a case where the user has selected to end shooting in response to the notification.

13. The image capturing apparatus according to claim 1, further comprising:
a changing unit configured to change the predetermined time period,
wherein in a case where the specific information has been set, the control unit performs control to prohibit changing of the predetermined time period.

14. A control method executed by an image capturing apparatus including an image capturing unit, comprising:
switching between ON and OFF of a medical mode of the image capturing apparatus, wherein the medical mode is an operation mode for shooting a medical image;
setting specific information;
associating the set specific information with an image that has been shot using the image capturing unit; and
performing, in a case where the medical mode is ON, control to
in a case where the specific information has not been set, cause the image capturing apparatus to transition to a power saving state when a state where a user has not operated the image capturing apparatus has continued for a predetermined time period, and
in a case where the specific information has been set, not cause the image capturing apparatus to transition to the power saving state even if the state where the user has not operated the image capturing apparatus has continued for the predetermined time period.

15. A non-transitory computer-readable storage medium which stores a program for causing an image capturing apparatus including an image capturing unit to execute a control method comprising:
switching between ON and OFF of a medical mode of the image capturing apparatus, wherein the medical mode is an operation mode for shooting a medical image;
setting specific information;
associating the set specific information with an image that has been shot using the image capturing unit; and
performing, in a case where the medical mode is ON, control to
capturing apparatus to transition to a power saving state when a state where a user has not operated the image capturing apparatus has continued for a predetermined time period, and
in a case where the specific information has been set, not cause the image capturing apparatus to transition to the power saving state even if the state where the user has not operated the image capturing apparatus has continued for the predetermined time period.
